(19) United States
(12) Patent Application Publication
(10) Pub. No.: US 2015/0209303 A1
(43) Pub. Date: Jul. 30, 2015

(54) ZEAXANTHIN FOR TUMOR TREATMENT

(71) Applicant: The New York Eye and Ear Infirmary, New York, NY (US)

(72) Inventors: Richard B. Rosen, New York, NY (US); Dan-Ning Hu, Garden City, NY (US)

(21) Appl. No.: 14/620,424

(22) Filed: Feb. 12, 2015

Related U.S. Application Data
(63) Continuation of application No. 13/840,970, filed on Mar. 15, 2013, now abandoned.
(60) Provisional application No. 61/645,010, filed on May 9, 2012.

(51) Int. Cl. A61K 31/047; A61K 45/06

(52) U.S. Cl. CPC A61K 31/047; A61K 45/06

(57) ABSTRACT

A composition comprising a pharmaceutically effective amount of zeaxanthin or its derivative for use in treating a malignant tumor and a method of using a pharmaceutically effective amount of zeaxanthin or its derivative either alone or together with one or more pharmaceutical agents for treating a malignant tumor. The tumor may be, but is not limited to breast cancer, cervix cancer, colon cancer, cutaneous melanoma, cutaneous squamous carcinoma, hepatocellular carcinoma, lung cancer, osteosarcoma, prostate cancer, and uveal melanoma. The pharmaceutically effective amount of zeaxanthin is generally above about 0.5 mg/kg/d to about 20 mg/kg/d.

### CROSS REFERENCE TO PRIOR APPLICATION

This application is a continuation application of U.S. application Ser. No. 13/840,970, filed Mar. 15, 2013, which claims priority to U.S. Provisional Patent Application No. 61/645,010, titled “Use of Zeaxanthin for Treating Tumors,” filed May 9, 2012.

### FIELD OF INVENTION

The present invention relates to methods of and compositions for treating tumors. In particular, the present invention relates to methods of and compositions for treating various tumors by administration of relatively high doses of zeaxanthin.

### BACKGROUND

Zeaxanthin belongs to the xanthophyll family of carotenoids and is one of the major components of the macular pigment in the retina of the eye. Zeaxanthin is an antioxidant and may have many physiological and pharmacological effects. Zeaxanthin is present in dark green, leafy vegetables, corn and egg yolks, and has been used for the prevention and treatment of several eye diseases such as age-related macular degeneration and also used as a nutrient ion supplement usually at a relatively low dosage, e.g., 1 mg zeaxanthin is used in the Ocuvite vitamin formula per capsule by Bausch and Lomb Inc.

Malignant tumors remain to be a major cause of death and drugs available for treating malignant tumors have been insufficient to meet the expectation of patients. New therapeutic compositions and therapeutic paradigms remain to be identified. For example, uveal melanoma is a common primary intraocular tumor in adults. Despite advances in diagnosis and treatment of uveal melanoma in the last decades, uveal melanoma still has a poor prognosis. Uveal melanoma-related mortality is 50% after 25 years. Systemic metastasis is common in uveal melanoma and the liver is the most frequent organ for metastasis. Most uveal melanoma patients with liver metastasis die within about 6 month and the median survival time after diagnosis of metastasis is only about 3.6 month. Because of the poor prognosis of metastatic melanoma, there is an urgent need for better therapies.

### SUMMARY OF THE INVENTION

In one aspect, the present invention is in part directed to a method of preventing the onset of a malignant tumor, delaying the progression of a malignant tumor, reducing cell viability of a malignant tumor, promoting cell apoptosis of a malignant tumor, or treating a malignant tumor in a subject by administering to said subject a composition comprising a pharmaceutically effective amount of zeaxanthin, or a zeaxanthin derivative. The malignant tumor may be breast cancer, cervix cancer, colon cancer, cutaneous melanoma, cutaneous squamous carcinoma, hepatocellular carcinoma, lung cancer, osteosarcoma, prostate cancer, or uveal melanoma.

In some embodiments, the pharmaceutically effective amount of zeaxanthin is about 0.5 mg/kg/d to about 20 mg/kg/d. In other embodiments, the pharmaceutically effective amount of zeaxanthin is about 2.5 mg/kg/d. The administration of zeaxanthin may be by an oral administration, a topical administration, a nasal administration, a rectal administration or an intravenous administration and the subject for administration may be a human individual, e.g., a patient.

In some embodiments, a pharmaceutically effective zeaxanthin derivative is used. The derivative may be a zeaxanthin derivative with an aldehyde, a dialdehyde, a ketone, a carboxylic acid, an epoxide, a furanoxide, a gamma-lactone, an alpha-hydroxy ketone, a diol, an acetal, a ketal, a halogenated derivative, an acetylated derivative, a derivative containing one or more alkynic bonds, and combination thereof. In some embodiments, the zeaxanthin or it derivative is a naturally occurring composition. In other embodiments, the zeaxanthin or derivative is synthetic or manmade.

In another aspect, the present invention is in part directed to a composition for preventing the onset of a malignant tumor, delaying the progression of a malignant tumor, reducing cell viability of a malignant tumor, promoting cell apoptosis of a malignant tumor, or treating a malignant tumor in a subject, which composition comprises a pharmaceutically effective amount of zeaxanthin or a zeaxanthin derivative.

In some embodiments, the composition is used to treat malignant tumors including breast cancer, cervix cancer, colon cancer, cutaneous melanoma, cutaneous squamous carcinoma, hepatocellular carcinoma, lung cancer, osteosarcoma, prostate cancer, and uveal melanoma. In some instances, the pharmaceutically effective amount of zeaxanthin used in these treatments is about 0.5 mg/kg/d (mg/kg/d refers to milligram per kilogram body weight per day) to about 20 mg/kg/d. In other instances, the pharmaceutically effective amount of zeaxanthin is about 2.5 mg/kg/d.

In some embodiments, the composition is administrated to the subject for preventing the onset of a malignant tumor, delaying the progression of a malignant tumor, reducing cell viability of a malignant tumor, promoting cell apoptosis of a malignant tumor, or treating a malignant tumor. The administration is an oral administration, a topical administration, a nasal administration, a rectal administration or an intravenous administration and the subject may be a human.

In some embodiments of the composition, a pharmaceutically effective amount of zeaxanthin derivative is used. The derivative may be a zeaxanthin derivative with an aldehyde, a dialdehyde, a ketone, a carboxylic acid, an epoxide, a furanoxide, a gamma-lactone, an alpha-hydroxy ketone, a diol, an acetal, a ketal, a halogenated derivative, an acetylated derivative, a derivative containing one or more alkynic bonds, and combination thereof.

In yet another aspect, the present invention is directed to a method of treating a malignant tumor in a patient by administering to the patient a pharmaceutically effective amount of zeaxanthin together with one or more pharmaceutical agents. The combination therapy may include zeaxanthin and one or more pharmaceutical agents that may be 5-Fluorouracil, Adriamycin, Cytoxan, Cisplatin, or Avastin®. In some embodiments, the malignant tumor subject to this combination therapy is a breast cancer, cervix cancer, colon cancer, cutaneous melanoma, cutaneous squamous carcinoma, hepatocellular carcinoma, lung cancer, osteosarcoma, prostate cancer, or uveal melanoma.

### BRIEF DESCRIPTION OF THE DRAWINGS

FIGS. 1A, 1B and 1C illustrate the dosage effects of zeaxanthin on normal uveal melanocytes (A) and uveal melanoma cells (SP6.5 cell line, B and C918 cell line, C). Cells were treated with zeaxanthin at various doses for 48 h, and cell survival was determined by 3-(4,5-dimethylthiazol-2-yl)-2,5-diphenyltetrazolium bromide (MTT) assay (absorbance at 540 nm). Data represent mean±SD from three independent experiments. *p<0.05 and **p<0.01, compared with the control group which are cells cultured without zeaxanthin.

FIGS. 2A, 2B and 2C illustrate the phase-contrast photomicrographs of uveal melanoma cells which have been treated with three different concentrations of zeaxanthin. Uveal melanoma cells (SP6.5 cell line) were seeded into 24-well plates and treated with zeaxanthin at 0 μM (2A), 30 μM (2B), and 100 μM (2C) levels for 48 hr.

FIG. 3 illustrates the fluorescence photomicrograph of pro-apoptotic effects of zeaxanthin on uveal melanoma cells after annexin V-FTIC staining Melanoma cells (SP6.5 cell line) were treated by zeaxanthin at a concentration of 100 μM for 24 hr, and the extent of apoptosis was determined by annexin V-FTIC staining. Cells were observed under fluorescence microscope using 492 nm and 530 nm filters for recognizing and detecting FITC. Apoptotic cells showed green staining of the cell membrane.

### DETAILED DESCRIPTION OF THE INVENTION

The present invention is partly based on the discovery that treatment with zeaxanthin can reduce the viability and induce apoptosis of malignant tumor cells, but has no effects on the viability and apoptosis of non-malignant tumor cells. As such, in one aspect, the invention is a method for treating malignant tumors using a pharmaceutically effective dose of zeaxanthin either alone or in combination with other therapeutic agents. In another aspect, the invention is a composition comprising a pharmaceutically effective dose of zeaxanthin for use in treating malignant tumors.

In some embodiments, other therapeutic agents that may be used in combination with zeaxanthin may be a conventional pharmaceutical agent, conventional anti-malignant tumor agent, additives, excipient, adjuvant and carriers. For example, a combination therapy may include zeaxanthin and one or more pharmaceutical agents including 5-Fluorouracil, Adriamycin, Cytoxan, Cisplatin, or Avastin®. Other anti-cancer agents as described in details on the website of National Cancer Institute under National Institutes of Health at http://www.cancer.gov/ may also be equally applied in a combination therapy with zeaxanthin.

As used herein, the word “treatment” is meant to include any intervention of the progress of a disease or other medical condition. The intervention of a malignant tumor may refer to the prevention of the onset of a malignant tumor, the delay of the progression of a malignant tumor already formed, the inhibition of malignant tumor cells, the promotion of malignant tumor cell apoptosis, or any other interference that can reduce malignant tumor or otherwise restore the diseased condition completely into a pre-diseased condition.

In some embodiments, the malignant tumor under treatment is breast cancer, cervix cancer, colon cancer, cutaneous melanoma, cutaneous squamous carcinoma, hepatocellular carcinoma, lung cancer, osteosarcoma, prostate cancer, or uveal melanoma. The pharmaceutically effective amount of zeaxanthin may differ in treating different malignant tumors because each malignant tumor may have different sensitivity to zeaxanthin. For example, as detailed in Example 1, A375 cell line of cutaneous melanoma is more sensitive to zeaxanthin with only 6% cells survive a treatment of 100 μM zeaxanthin. In contrast, DU-145 cell line of prostate cancer is less sensitive to zeaxanthin with about 41% cells survive the same treatment of 100 μM zeaxanthin.

In some embodiments, the pharmaceutically effective amount of zeaxanthin administrated for treating malignant tumors in a subject is about 0.5 mg/kg/d to about 20 mg/kg/d. In other embodiments, the pharmaceutically effective amount of zeaxanthin is about 1.0 mg/kg/d to about 5.0 mg/kg/d. In some instance, the pharmaceutically effective amount of zeaxanthin is about 2.5 mg/kg/d. In other instances, the pharmaceutically effective amount of zeaxanthin is about 1.5 mg/kg/d. The dosage of zeaxanthin may be determined by a doctor and may vary in accordance to many factors including, but not limited to, the type of malignant tumor, the delivery method, the purpose of treatment, and the method of treatment, e.g., a combination therapy with other anti-cancer agents versus zeaxanthin alone.

The subject for administration of zeaxanthin may be a human individual. In some embodiments, the individual may be a seemingly normal individual without apparent clinical manifestation of malignant tumors but may have a suspected pathological manifestation of malignant tumors. In these individuals, the treatment with zeaxanthin may be for the purpose of preventing the formation of malignant tumors. In other embodiments, the individual may already be diagnosed with malignant tumors, and the treatment with zeaxanthin may be for the purpose of reducing the malignant tumor cell viability. As detailed in Example 2, to treat diagnosed malignant tumor patients, zeaxanthin may be used in combination with other anti-tumor pharmaceutical agents, i.e., combination therapy. In a combination therapy, pharmaceutical agents may, act together in reducing tumor cell viability, promoting tumor cell apoptosis, or inhibiting tumor cell proliferation, whether synergistically or not.

In addition to varied dosage per administration, the frequency of delivery may also vary depending on a treatment plan designed by a doctor. The severity of malignant tumor in a subject may require more frequent treatment to maintain the continuous contact of the drug with malignant tumor cells. On the other hand, if a treat is only for preventing malignant tumor formation, a continuous contact of the drug with cells may not be desirable and the frequency may be reduced for that purpose.

The method of administering a composition in treating malignant tumors may vary and the choice is partly based on the efficiency and safety of the method in delivering the composition to the target tumor cells. For example, for systematic delivery of the composition, the administration of zeaxanthin may be by an oral administration. For delivering the composition to skin cells or cells capable of absorbing topical compositions, a topical administration may be preferred. In other instances, a nasal administration may be preferred to deliver zeaxanthin to the oral cavity or to brain cells by crossing the blood-brain barrier. In yet other instances, a rectal administration is preferred to deliver zeaxanthin to lower digestive tracts. In yet other instances, an intravenous administration is preferred to quickly deliver zeaxanthin systematically.

In other embodiments, a pharmaceutically effective zeaxanthin derivative is used instead of or in addition to zeaxanthin. Such derivatives may be desirable when zeaxanthin is found to be not ideally effective to certain malignant tumors or safe to certain patient population. These desirable derivatives are ideally more effective or less toxic to those certain malignant tumors. The derivative may be a zeaxanthin derivative in the form of an aldehyde, a dialdehyde, a ketone, a carboxylic acid, an epoxide, a furanoxide, a gamma-lactone, an alpha-hydroxy ketone, a diol, an acetal, a ketal, a halogenated derivative, an acetylated derivative, a derivative containing one or more alkynic bonds, and combination thereof.

In some embodiments, zeaxanthin may be natural products extracted from many different organisms. For example, zeaxanthin may be extracted from paprika or marine bacteria Olleya marilimosa. See U.S. Pat. Nos. 5,854,015 and 5,747,544, and U.S. Patent Publication No. 2012/0095108, which patents and publication are hereby incorporated by reference in its relevant parts related to zeaxanthin preparation, isolation and purification. In other embodiments, the zeaxanthin or its derivative is synthetic or manmade.

The compositions comprising zeaxanthin that are used to treat malignant tumors may be formulated in various forms. Illustrative, non-limiting examples are soft and hard capsules, gel, pellet, tablet, granule, grains, powder, and liquid formulation such as suspensions. In addition, the formulation may be a slow release form that may have a prolonged time before dosages so that the frequency of drug delivery may be reduced.

It should be understood that the term “tumor” refers to any uncontrolled growth of cells in a body of an animal. The term “malignant tumor” is herein used interchangeably with the term “cancer”, which refers to diseases where abnormal cells divide without control.

It should be understood that this invention is not limited to the particular methodologies, protocols and reagents, described herein, which may vary. The terminology used herein is for the purpose of describing particular embodiments only, and is not intended to limit the scope of the present invention, which is defined solely by the claims.

Examples of the disclosed subject matter are set forth below. Other features, objects, and advantages of the disclosed subject matter will be apparent from the detailed description, figures, examples and claims. Methods and materials substantially similar or equivalent to those described herein can be used in the practice or testing of the presently disclosed subject matter. Exemplary methods and materials are now described as follows.

### Example 1

### Cytotoxic Effects of Zeaxanthin on Malignant Tumor Cells

In this Example, we were able to demonstrate that zeaxanthin had cytotoxic effects on at least 12 types of malignant tumors but did not have cytotoxic effects on non-tumor cells. The 12 types of malignant tumors that were tested in this example were uveal melanoma including 4 cell lines, SP6.5, M17, OCM-2 and C918; cutaneous melanoma including 2 cell lines, A375 and COLO 829; hepatocellular carcinoma including 1 cell line Hep G2; cervix cancer including 1 cell line HeLa; cutaneous squamous carcinoma including 1 cell line, SCC1; osteosarcoma including 1 cell line, OS143; pancreatic cancer including 1 cell line, PANC; lymphoma cell line including 1 cell line, BC-1; lung cancer including 4 cell lines, A549, NCI-H1755, NCI-H358 and NCI-H820; colon cancer including 3 cell lines, Lovo, HCT-15 and HCT-116; prostate cancer including 2 cell lines, PC-3 and DU-145; and breast cancer including 3 cell lines, T-47D, MDA and MCF7.

All the malignant tumor cell lines except the four uveal melanoma cell lines were obtained from the American Type Culture Collection. The four uveal melanoma cell lines are each described in the published references as follows: (i) the human M17 uveal melanoma cell line in Hu et al., Ocular Oncology. New York, N.Y.: Marcel Dekker; 2003; 189-210; (ii) the SP6.5 uveal melanoma cell line in Soulieres et al., Int J Cancer. 1991; 49:498-503; (iii) the OCM-2 uveal melanoma cell line in Kan-Mitchell et al., Invest Ophthalmol Vis Sci. 1989 May; 30(5):829-34; and the C918 uveal melanoma cell line in Daniels et al., Lab. Invest. 1996; 75:55-66.

Three non-tumor or normal cell lines were used as the controls. The three non-tumor cell lines included one human fibroblasts cell line isolated from donor skin, one human normal uveal melanocyte cell line and one human normal retinal pigment epithelial (RPE) cells isolated from donor eyes. See Hu et al., Invest Ophthalmol Vis Sci. 1993; 33:2210-2219; and Wu et al., Mol Vis 2010; 16:1864-73. The non-tumor three cell lines were primary culture and mortal cell lines, which could be cultured in vitro and divide about 20-30 times. The non-tumor three cell lines used in the test were in the early passages with doubling time (DOT) at about 2.0-2.5 days.

Each malignant tumor cell line was cultured in Dulbecco's modified Eagle's medium (DMEM, GIBCO, Carlsbad, Calif.) supplemented with 10% fetal bovine serum (FBS, GIBCO). Cells were incubated in a humidified 5% CO₂ atmosphere at 37° C. After reaching confluence, cells were detached by trypsin-EDTA solution (GIBCO), diluted 1:4-1:6, plated for subculture and were passaged routinely at a dilution of 1:4-1:6 every 4-6 days.

Normal RPE cells and fibroblasts were cultured similar to the malignant tumor cells. Uveal melanocytes were cultured with F12 medium supplemented with 10% FBS, 2 mM glutamine, 20 ng/ml bFGF, 0.1 mM isobutylmethylxanthine, 10 ng/ml cholera toxin (F12 medium from GIBCO, Invitrogen, Carslbad, Calif., USA; all others from Sigma, St. Louis, Mo., USA) as we reported previously (Hu et al., Invest Ophthalmol Vis Sci. 1993; 33:2210-2219).

Zeaxanthin was provided by ZeaVision LLC and was dissolved in DMSO at a concentration of 60 mM as a stock solution. The stock solution was dissolved to the culture medium to obtain the final concentrations of zeaxanthin at 10, 30 and 100 μM (which would result in DMSO at concentrations of 0.017%, 0.05% and 0.17% in the culture media, respectively) in the tests on their cytotoxic effects in malignant tumor cell lines and extended to 300 μM (which resulted in DMSO concentration at 0.5% in the culture media) in normal cell lines. A high concentration of DMSO in cell culture media may affect cell viability.

Specifically, cells were plated in 96 well plates at a density of 5×10³ cells per well. After incubation for 24 hr, zeaxanthin was added to the wells to achieve various final concentrations (10, 30 and 100 μM; extended to 300 μM in normal cells) and cultured for 48 hours. Then 50 μl of tetrazolium bromide, 3-(4,5-dimethylthiazol-2-yl)-2,5-diphenyltetrazolium bromide (MTT, 1 mg/ml, SIGMA) was added to each well and incubated for 4 hr. The medium was withdrawn and 100 μl of DMSO (SIGMA) was added into each well. The optical density was read at 540 nm using a microplate reader (Multiskan EX, Thermo, Vantaa, Finland). The controls were cells cultured without zeaxanthin but with DMSO concentrations at 0.17% in malignant tumor cell lines which corresponded to the concentration of DMSO in the samples treated with 100 μM zeaxanthin, or at 0.5% in non-tumor cell lines which corresponded to the concentration of DMSO in the samples treated with 300 μM zeaxanthin. All cell lines were tested in triplicates. See Lu et al., Curr Eye Res. 2010; 35:352-360.

The optical densities were measured and subjected to statistical analysis. Statistical analysis was performed using the ANOVA one-way test in comparing data from tested group to the controls. A difference at p<0.05 was considered to be statistically significant.

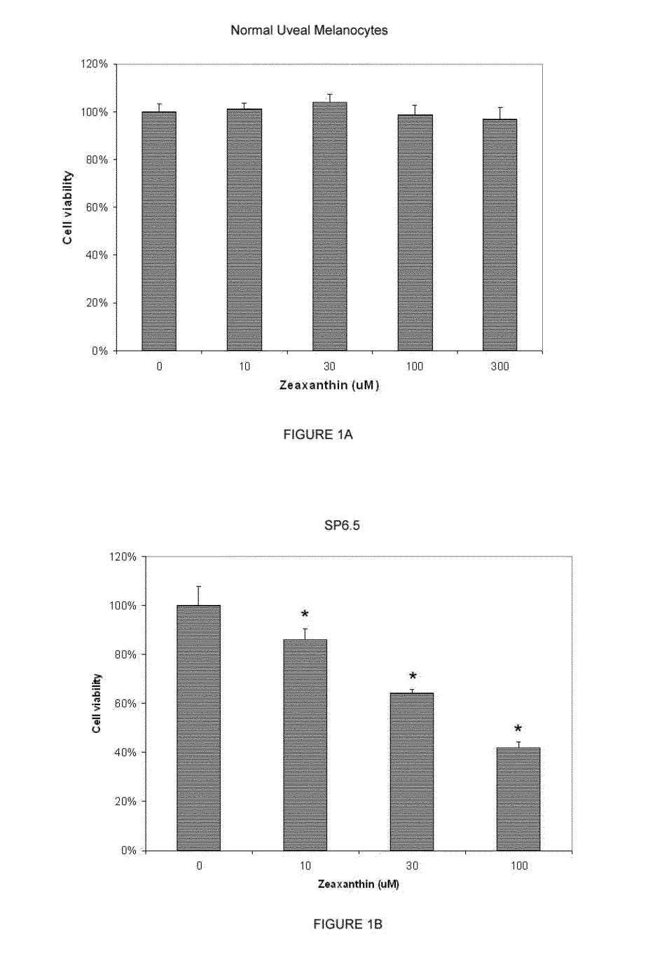

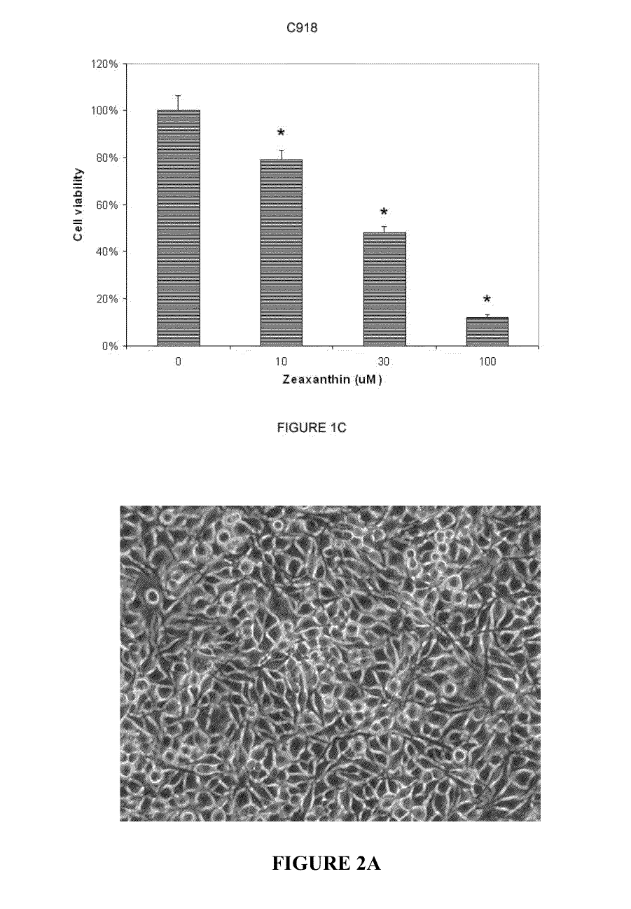

In the three non-tumor cell lines tested with zeaxanthin at final concentrations of 10, 30, 100 and 300 μM, none of them showed a statistically significant difference in cell viability as compared to the control cells which were not treated with zeaxanthin. See Table 1 for the measured viability result in each cell group. Therefore, a final concentration of zeaxenthin as high as 300 μM did not affect cell viability of any of the three non-tumor cell lines. A representative chart in FIG. 1A shows that no significant difference in cell viability could be detected in each group of cells that were treated with zeaxanthin at final concentrations of 10, 30, 100, or 300 μM in comparison to the cell without any zeaxanthin treatment.

TABLE 1
Cell viability of non-tumor cell lines treated with zeaxanthin

| Non-tumor | Cell viability | | | | | |
|---|---|---|---|---|---|---|
| Cell line | 0 (M ± SD) | 10 μM | 30 μM | 100 μM | 300 μM | ID50 |
| Uveal | 100 ± 3.4% | 101 ± 2.8% | 1.4 ± 3.3% | 99 ± 3.9% | 97 ± 4.6% | >300 μM |
| malenocyte | | | | | | |
| RPE | 100 ± 4.1% | 104 ± 3.7% | 97 ± 4.2% | 101 ± 2.4% | 95 ± 2.2% | >300 μM |
| Fibroblast | 100 ± 2.6% | 1.5 ± 4.4% | 102 ± 4.7% | 99 ± 3.7% | 97 ± 2.8% | >300 μM |

As shown in Table 2, we demonstrated that zeaxanthin significantly reduced cell viability at a low dosage level (at a final concentration of 10 μM or less) and the ID50 (the final concentration of zeaxanthin that would kill 50% of cells in the culture after treatment) was less than 100 μM in the cell lines. The cell lines in Table 2 are hereby referred to as zeaxanthin sensitive cell lines. These zeaxanthin sensitive cell lines were hepatocellular carcinoma (1 cell line, Hep G2), colon cancer (all 3 cell lines tested, Lovo, HCT-15 and HCT-116), prostate cancer (both 2 cell lines tested, PC-3 and DU-145), lung cancer (2 in 4 cell lines tested, NCI-H358 and NCI-H820), cutaneous melanoma (both 2 cell lines tested, A375, COLO 829), uveal melanoma (all 4 cell lines tested, SP6.5, M17, OCM-2 and C918), cervix cancer (1 cell line, HeLa), breast cancer (1 in 3 cell lines tested, T-47D), cutaneous squamous carcinoma (1 cell line, SCC1) and osteosarcoma (1 cell line, OS143).

A representative chart in FIG. 1B depicts the cell viability test results for the SP6.5 cell line treated with zeaxanthin at final concentrations of 0, 10, 30, or 100 μM. SP6.5 cells that were treated with zeaxanthin at a final concentration of 10 μM, 30 μM, and 100 μM showed significantly less viability than the control cells without any zeaxanthin treatment and showed a reduction of cell viabilities of about 14% (P<0.05), 36% (P<0.01), and 58% (P<0.01), respectively.

A representative chart in FIG. 1C depicts the cell viability test results for the C918 cell line treated with zeaxanthin at final concentrations of 0, 10, 30, or 100 μM. C918 cells that were treated with zeaxanthin at a final concentration of 10 μM, 30 μM, and 100 μM showed significantly less viability than the control cells without any zeaxanthin treatment and showed a reduction of cell viabilities of about 21% (P<0.01), 52% (P<0.01), and 88% (P<0.01), respectively.

Similar to the test results in SP6.5 and C918 cell lines, cell viability in each of the zeaxanthin sensitive cell lines in Table 2 could be suppressed by zeaxanthin at a concentration as low as 10 μM. As shown in the column “10 μM”, when cells were treated with zeaxanthin at a final concentration of 10 μM, cell viability decreased significantly in the range of 62% to 90% in comparison to the cells without zeaxanthin treatment. In addition, as shown in the column “30 μM”, when cells were treated with zeaxanthin at a final concentration of 30 μM, cell viability decreased significantly in the range of 41% to 78% in comparison to the cells without zeaxanthin treatment. Further, as shown in the column “100 μM”, when cells were treated with zeaxanthin at a final concentration of 100 μM, cell viability decreased significantly in the range of 6% to 46% in comparison to the cells without zeaxanthin treatment. Finally, the ID50 value for each cell line was in the range of 21.4 to 88.3.

TABLE 2
Cell viability of various malignant tumors treated
with zeaxanthin in zeaxanthin sensitive cell lines.

| | | Cell viability | | | | |
|---|---|---|---|---|---|---|
| Tumor | Cell line | 0 (M ± SD) | 10 μM | 30 μM | 100 μM | ID50 |
| Cervix cancer | HeLa | 100 ± 4.7% | 66 ± 3.3%** | 46 ± 1.6%** | 7.9 ± 2.0%** | 26.0 μM |
| Cutaneous | A375 | 100 ± 6.5% | 62 ± 2.7%** | 41 ± 2.7%** | 6.0 ± 1.1%** | 21.4 μM |
| Melanoma | | | | | | |
| | COLO829 | 100 ± 6.8% | 73 ± 4.6%** | 52 ± 2.0%** | 28 ± 1.2%** | 35.8 μM |
| Colon Cancer | HCT-15 | 100 ± 8.2% | 62 ± 3.4%** | 46 ± 2.4%** | 12 ± 1.3%** | 25.0 μM |
| | LoVo | 100 ± 5.5% | 76 ± 3.8%** | 51 ± 2.5%** | 32 ± 1.6%** | 33.7 μM |
| | HCT116 | 100 ± 7.6% | 67 ± 3.7%** | 56 ± 2.3%** | 16 ± 1.4%** | 37.4 μM |
| Uveal | C918 | 100 ± 6.4% | 79 ± 4.1%** | 48 ± 2.6%** | 12 ± 1.0%** | 28.7 μM |
| melanoma | | | | | | |
| | SP6.5 | 100 ± 7.6% | 86 ± 4.3%* | 64 ± 1.7%** | 42 ± 2.2%** | 75.7 μM |
| | M17 | 100 ± 5.7% | 84 ± 3.9%** | 69 ± 2.5%** | 41 ± 1.0%** | 77.5 μM |
| | OCM2 | 100 ± 4.4% | 90 ± 4.9%* | 70 ± 2.1%** | 46 ± 2.0%** | 88.3 μM |
| Breast cancer | T-47D | 100 ± 6.3% | 83 ± 4.2%** | 57 ± 3.5%** | 31 ± 1.4%** | 48.8 μM |
| Skin cancer | SCC1 | 100 ± 5.4% | 80 ± 3.5%** | 65 ± 2.9%** | 13 ± 1.4%** | 50.2 μM |
| Liver cancer | HepG2 | 100 ± 5.0% | 79 ± 3.7%** | 58 ± 1.8%** | 32 ± 1.2%** | 51.5 μM |
| Prostate | PC-3 | 100 ± 9.3% | 74 ± 4.4%** | 62 ± 3.2%** | 39 ± 2.6%** | 66.5 μM |
| cancer | | | | | | |
| | DU-145 | 100 ± 9.5% | 78 ± 5.0%** | 65 ± 5.5%** | 41 ± 2.6%** | 73.8 μM |
| Lung cancer | NCI-H358 | 100 ± 5.1% | 80 ± 3.5%** | 65 ± 3.0%** | 37 ± 1.3%** | 67.5 μM |
| | NCI-H820 | 100 ± 7.5% | 76 ± 4.5%** | 68 ± 3.1%** | 38 ± 1.7%** | 72.0 μM |
| Osteosarcoma | OS143 | 100 ± 6.9% | 82 ± 5.0%** | 78 ± 3.4%** | 42 ± 1.3%** | 84.4 μM |

*0.01 < P < 0.05, compared with the controls.
**P < 0.01, compared with the controls.

In the six cell lines as shown in Table 3, we found that higher dosage levels of zeaxanthin, e.g., 30 μM or 100 μM, were required to significantly reduce cell viability. These six cell lines are hereby referred to as zeaxanthin insensitive cell lines. These zeaxanthin insensitive cell lines were from pancreatic cancer (1 cell line, PANC), lung cancer (2 cell lines, A549 and NCI-H1755), breast cancer (2 cell lines, MDA and MCF7), and lymphoma (1 cell line, BC-1). In NCI-H1755, MCF-7, and BC-1 cell lines, when the final concentration of zeaxanthin was 30 μM or more, cell viability decreased significantly in comparison to cell without zeaxanthin treatment. In contrast, in PANC and A549 cell lines, when the final concentration of zeaxanthin was 100 μM or more, cell viability decreased significantly in comparison to cell without zeaxanthin treatment. However, in MDA cell line, we did not see statistically significant decrease of cell viability after zeaxanthin treatment even at a final concentration as high as 100 μM. In each of the six cell lines, ID50 was over 100 μM.

The testing results in Tables 2 and 3 show that zeaxanthin can be applied to reduce cell viability of malignant tumor cells and thereby inhibits tumor cell growth at an appropriate dosage. For the zeaxanthin sensitive cell lines, the viability of malignant tumor cells treated with an amount of zeaxanthin at a final concentration of 10 μM is significantly lower statistically than that in the controls. For the zeaxanthin insensitive cell lines, the viability of malignant tumor cells treated with an amount of zeaxanthin at a final concentration of 30 μM or 100 μM is significantly lower than that in the controls.

TABLE 3
Cell viability of various malignant tumors treated with
zeaxanthin in zeaxanthin insensitive cell lines

| | | Cell viability | | | | |
|---|---|---|---|---|---|---|
| Tumor | Cell line | 0 (M ± SD) | 10 μM | 30 μM | 100 μM | ID50 |
| Lung cancer | A549 | 100 ± 7.9% | 94 ± 6.6% | 86 ± 4.4% | 67 ± 6.1%** | >100 μM |
| | NCI-H1755 | 100 ± 8.0% | 86 ± 6.7% | 81 ± 4.5%* | 81 ± 90%* | >100 μM |
| Breast cancer | MDA | 100 ± 9.4% | 97 ± 6.36% | 95 ± 5.1% | 92 ± 9.2% | >100 μM |
| | MCF-7 | 100 ± 6.6% | 86 ± 4.0%* | 73 ± 8.3%** | 52 ± 2.4%** | >100 μM |
| Pancreas | PANC-1 | 100 ± 6.6% | 101 ± 6.0% | 98 ± 5.5% | 76 ± 5.1%** | >100 μM |
| cancer | | | | | | |
| Lymphoma | BC-1 | 100 ± 9.5% | 79 ± 8.2% | 67 ± 11%* | 69 ± 7.1%* | >100 μM |

*0.01 < P < 0.05, compared with the controls.
**P < 0.01, compared with the controls.

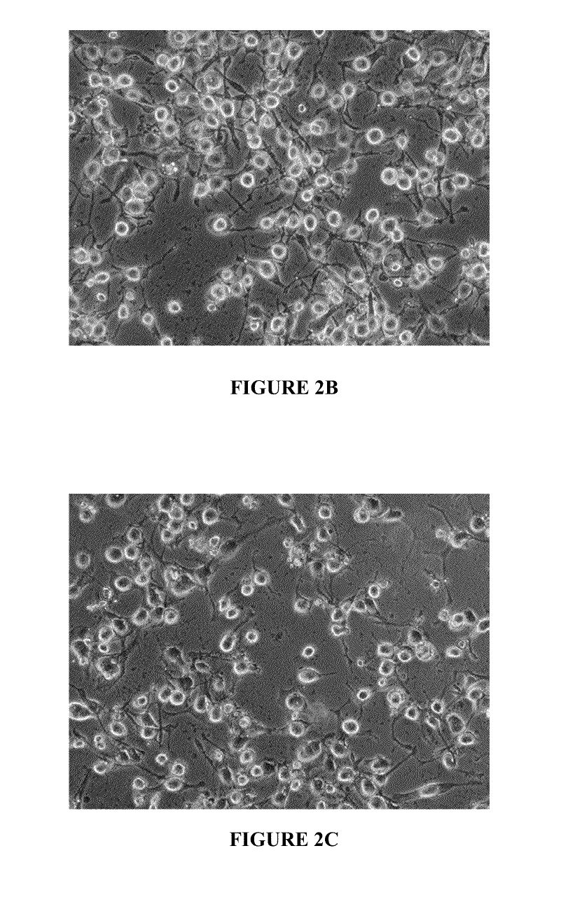

Viability of cells treated with zeaxanthin was also visualized in the following tests. In these tests, the same amount of uveal melanoma cells were cultured and treated with zeaxanthin at different concentrations, and the treated cells were examined under microscope. Specifically, the uveal melanoma cell line SP6.5 was seeded into 24-well plates and treated with zeaxanthin at final concentrations of 0 μM, 30 μM, and 100 μM for 48 hr. As shown in FIGS. 2A, 2B and 2C, as the concentration of zeaxanthin increased from 0 μM, to 30 μM, and to 100 μM, the number of cells in the culture that could be seen under microscope decreased progressively. FIG. 2A shows a representative photographic capture of cells that were not treated with zeaxanthin. FIG. 2B shows a representative photographic capture of cells that were treated with zeaxanthin at a final concentration of 30 μM. FIG. 2C shows a representative photographic capture of cells that were treated with zeaxanthin at a final concentration of 100 μM. The number of cells alive in FIG. 2A is more than that in FIG. 2B, which in turn is more than that in FIG. 2C. The decreasing number of live cells in cultures treated with increasing amount of zeaxanthin again demonstrated the use of zeaxanthin in inhibiting tumor cell growth and/or suppressing tumor cell division.

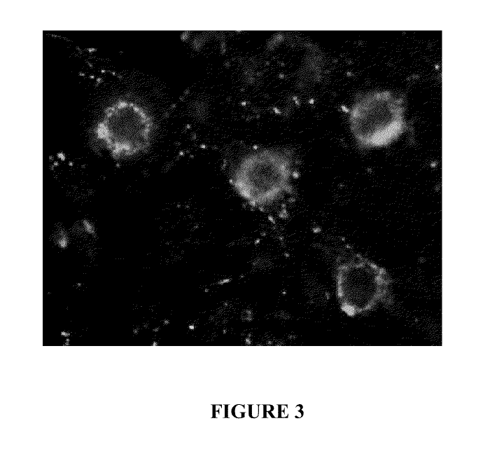

We also showed that zeaxanthin treatment could induce apoptosis of malignant tumor cells in the following tests. In these tests, we measured the extent of apoptosis of cells treated with zeaxanthin by annexin V-FITC staining analysis. Specifically, uveal melanoma cells (SP6.5) were plated into 8-well plates at a density of 6×10⁴ cells/well. After culturing for 24 hours, cells were treated with zeaxanthin at final concentration of 10, 30, and 100 μM for another 24 hours in a CO₂ incubator at 37 C. Then the culture medium was aspirated and the cells were washed with the binding buffer in preparation for annexin V-FITC staining analysis. FITC-annexin V was added into each well and the cells were stained for 15 min in the dark, followed by washing with the binding buffer. The cells were then observed under fluorescence microscope using 492 nm and 530 nm filters that facilitate FITC fluorescence detection to measure the extent of apoptosis based on the principle that cell membranes of apoptotic cells were stained bright green in color. As a result, we observed the extent of apoptosis in uveal melanoma cells induced by zeaxanthin in a dose-dependent manner. The apoptosis became As shown in FIG. 3, in cells treated with 100 μM zeaxanthin, numerous cells showed staining of the cell membrane with annexin V and appeared bright green in color (the bright green is shown as bright white in FIG. 3). The presence of bright green color indicated that they were apoptotic cells.

The above cell viability and the apoptosis assay results demonstrated that zeaxanthin treatment could significantly decrease cell viability and increase apoptosis in the malignant tumor cells that were treated with zeaxanthin at a pharmaceutically appropriate concentration. For example, zeaxanthin treatment at a concentration as low as 10 μM could reduce viability and induce apoptosis of uveal melanoma cells.

In contrast, the viability and apoptotic activities of non-tumor or normal uveal melanocytes, retinal pigment epithelial cells, and fibroblasts were not affected by zeaxanthin even at a concentration as high as 300 μM. See Table 1 for test results on non-tumor cells treated with zeaxanthin. As such, malignant tumor cells are sensitive to zeaxanthin treatment whereas normal uveal melanocytes, retinal pigment epithelial cells and fibroblasts are resistant to zeaxanthin treatment. Therefore, the therapeutic effects of zeaxanthin are selectively targeted to some malignant tumor cells but not to non-tumor or normal cells.

### Example 2

### Use of Zeaxanthin in Treating Malignant Hepatic Tumor

In this example, zeaxanthin was used to treat a patient with malignant hepatic tumor. The patient, an 80 years old Caucasian, who had been treated for neovascular age-related macular degeneration, was diagnosed with a malignant hepatic tumor and underwent an extensive 12-hour resection. The tumor recurred in a few months in the patient after resection. The patient was then treated with zeaxanthin in view of the extensive and safe use of zeaxanthin as a supplement of the treatment of macular disorders.

In treating the recurred malignant hepatic tumor in the patient, we prescribed a dose of 80 mg/day of zeaxanthin which was used to supplement his conventional treatment of Avastin® infusion. After several months, the patient's condition remained stable and subsequent Magnetic Resonance Imaging (MRI) scans showed that the multiple recurrent tumors began to shrink significantly to the point of where the tumors could not be detected. The patient's condition continued to improve under the combination therapy of zeaxanthin and Avastin® infusion in the next 9 months. The patient was able to resume his social life during the period of 9 months and then passed away after 2 months. The patient's life was prolonged more than 1 year following his projected mortality under the conventional Avastin® infusion treatment on the recurring malignant hepatic tumor.

Therefore, zeaxanthin showed anti-tumor effects on malignant hepatic tumor cells in a human patient. A dosage of 80 mg/day in the patient whose weight at the time was about 68 kg showed efficacy in reducing the hepatic tumor in combination with the conventional treatment. Therefore, a dosage of about 1.2 mg/kg/d of zeaxanthin was sufficient to help reducing tumor cell viability in the patient under the combination therapy.

While the present invention has been disclosed with reference to certain embodiments, numerous modifications, alterations, and changes to the described embodiments are possible without departing from the full scope of the invention, as described in the appended specification and claims.

What we claim are:

1. A method of preventing the onset of a malignant tumor, delaying the progression of a malignant tumor, reducing cell viability of a malignant tumor, promoting cell apoptosis of a malignant tumor, or treating a malignant tumor in a subject by administering to said subject a composition comprising a pharmaceutically effective amount of zeaxanthin, or a zeaxanthin derivative.

2. The method of claim 1, wherein said malignant tumor is a malignant tumor selected from the group consisting of breast cancer, cervix cancer, colon cancer, cutaneous melanoma, cutaneous squamous carcinoma, hepatocellular carcinoma, lung cancer, osteosarcoma, prostate cancer, and uveal melanoma.

3. The method of claim 2, wherein the pharmaceutically effective amount of zeaxanthin is about 0.5 mg/kg/d to about 20 mg/kg/d.

4. The method of claim 2, wherein the pharmaceutically effective amount of zeaxanthin is about 2.5 mg/kg/d.

5. The method of claim 1, wherein the administration is an oral administration, a topical administration, a nasal administration, a rectal administration or an intravenous administration.

6. The method of claim 1, wherein the subject is a human.

7. The method of claim 1, wherein the zeaxanthin derivative is a derivative selected from the group consisting of an aldehyde, a dialdehyde, a ketone, a carboxylic acid, an epoxide, a furanoxide, a gamma-lactone, an alpha-hydroxy ketone, a diol, an acetal, a ketal, a halogenated derivative, an acetylated derivative, a derivative containing one or more alkynic bonds, and combination thereof.

8. The method of claim 1, wherein the zeaxanthin is a naturally occurring zeaxanthin or a synthetic zeaxanthin.

9. A composition for preventing the onset of a malignant tumor, delaying the progression of a malignant tumor, reducing cell viability of a malignant tumor, promoting cell apoptosis of a malignant tumor, or treating a malignant tumor in a subject, said composition comprising a pharmaceutically effective amount of zeaxanthin or a zeaxanthin derivative.

10. The composition of claim 9, wherein said malignant tumor is a malignant tumor selected from the group consisting of breast cancer, cervix cancer, colon cancer, cutaneous melanoma, cutaneous squamous carcinoma, hepatocellular carcinoma, lung cancer, osteosarcoma, prostate cancer, and uveal melanoma.

11. The composition of claim 10, wherein the pharmaceutically effective amount of zeaxanthin is about 0.5 mg/kg/d to about 20 mg/kg/d.

12. The composition of claim 10, wherein the pharmaceutically effective amount of zeaxanthin is about 2.5 mg/kg/d.

13. The composition of claim 9, wherein the composition is administrated to the subject for preventing the onset of a malignant tumor, delaying the progression of a malignant tumor, reducing cell viability of a malignant tumor, promoting cell apoptosis of a malignant tumor, or treating a malignant tumor.

14. The composition of claim 13, wherein the administration is an oral administration, a topical administration, a nasal administration, a rectal administration or an intravenous administration.

15. The composition of claim 9, wherein the subject is a human.

16. The composition of claim 9, wherein the zeaxanthin derivative is a derivative selected from the group consisting of an aldehyde, a dialdehyde, a ketone, a carboxylic acid, an epoxide, a furanoxide, a gamma-lactone, an alpha-hydroxy ketone, a diol, an acetal, a ketal, a halogenated derivative, an acetylated derivative, a derivative containing one or more alkynic bonds, and combination thereof.

17. The composition of claim 9, wherein the zeaxanthin is a naturally occurring zeaxanthin or is a synthetic zeaxanthin.

18. A method of treating a malignant tumor in a patient by administering to the patient a pharmaceutically effective amount of zeaxanthin together with one or more pharmaceutical agents.

19. The method of claim 18, wherein the one or more pharmaceutical agents comprising 5-Fluorouracil, Adriamycin, Cytoxan, Cisplatin, or Avastin®.

20. The method of claim 18, wherein said malignant tumor is a malignant tumor selected from the group consisting of breast cancer, cervix cancer, colon cancer, cutaneous melanoma, cutaneous squamous carcinoma, hepatocellular carcinoma, lung cancer, osteosarcoma, prostate cancer, and uveal melanoma.
